(19) United States
(12) Patent Application Publication
(10) Pub. No.: US 2006/0129069 A1
(43) Pub. Date: Jun. 15, 2006

(54) Apparatus and method for pain measurement

(76) Inventors: A. Vania Apkarian, Chicago, IL (US); Dante R. Chialvo, Chicago, IL (US)

Correspondence Address: OPPEDAHL & OLSON LLP, P.O. BOX 5388, DILLON, CO, 80435-5388, US

(21) Appl. No.: 10/906,125

(22) Filed: Feb. 3, 2005

Related U.S. Application Data
(60) Provisional application No. 60/593,132, filed on Dec. 14, 2004.

(51) Int. Cl. A61B 5/103; A61B 5/00

(52) U.S. Cl. 600/595; 600/555

(57) ABSTRACT

Time-series of pain ratings are analyzed from patients with CLBP, PHN, and from normal subjects imagining back pain. A non-linear technique is used to determine whether each time-series examined is scale free, and derives a single parameter with which each time-series is characterized, permitting a contrast between pain conditions.

### CROSS-REFERENCE TO RELATED APPLICATIONS

This application claims priority from U.S. application No. 60/593,132, filed Dec. 14, 2004, which application is incorporated herein by reference for all purposes.

### BACKGROUND

Measurement of pain remains subjective and difficult to quantify. Currently the only objective approach for establishing presence of pain entails examining brain activity. By definition, chronic pain is a state of ongoing pain for long periods after the healing associated with the initial pain-inciting event. According to the International Association for the Study of Pain (IASP), pain is defined as “an unpleasant sensory and emotional experience associated with actual or potential tissue damage, or described in terms of such damage,” with the added note that “pain is always subjective” (Merskey and Bogduk, 1994). Since pain is often reported in the absence of obvious tissue damage, according to IASP, there is usually no way to distinguish between ‘psychological’ experiences of pain from that due to tissue damage. As a result subjective reporting remains fundamental for documenting presence of pain. Of course new advances in human brain imaging techniques now provide direct, objective, counterpart to this subjectivity and indicate that the experience of pain is associated with a well-defined pattern of brain activity. This relationship has been established mainly for acute, experimental settings, studying healthy subjects' brain activity in response to painful stimuli. The present invention addresses the issue of characterizing pain in the absence of a painful stimulus.

Acute pain is short lasting and properly treated with various types of analgesics. In contrast, chronic pain is defined as pain that persists post healing (operationally defined as persisting for longer than 3-6 months after healing), and current understanding of physiological mechanisms underlying such conditions remains rudimentary. Chronic pain impacts a large portion of society. Recent surveys estimate that about 10% of adults suffer from severe chronic pain, and in majority of such cases, including two highly prevalent conditions, post herpetic neuropathy (PHN) and chronic low back pain (CLBP), therapeutic approaches are not validated scientifically and responses to medications are highly variable. A fundamental property of chronic pain is the presence of spontaneous pain, and its presence is the main reason for subjects seeking medical care. Clinicians agree that the incidence of spontaneous pain is very high in chronic pain; yet the present applicant could find only a single source estimating this incidence: 98.6% in chronic pain conditions of central origin. Traditionally, clinical pain conditions have been contrasted by questionnaires. This approach, however, is not adequate to study dynamical properties.

Past efforts to provide objective measurement of pain include:

US 6018675—Assembly and method for objectively measuring pain in a subject

US 5941833—Heatbeam dolorimeter for pain and sensory evaluation

US 4007420—Device for measuring a nociceptive reaction of laboratory animals

US 20020107434—Objective pain measurement system and method

US 20020086343—Methods and compositions for pain management

WO 03/036261—Drug development by rapid neuroimaging of neural cells; and

WO 01/74240—Method and apparatus for objectively measuring pain, pain treatment and other related techniques.

It would be extremely desirable if methods and apparatus could be divised which would permit simple, reliable objective measurement of pain in human patients.

### SUMMARY OF THE INVENTION

So far as the present applicants are aware, temporal characteristics of spontaneous pain have remained unexplored. Unlike other sensory modalities, fluctuations in intensity of pain are slow and highly salient, and as a result patients, and normal subjects, can readily indicate their level of pain on a continuous time-scale. Empirical observations by the applicants demonstrate that intensity of chronic pain fluctuates spontaneously, and subjects instructed to indicate their pain intensity on a continuous scale comply readily. This approach allows gathering information about ongoing perceived pain over time and enables investigation of underlying dynamical processes. The present invention analyzes time-series of pain ratings from patients with CLBP, PHN, and from normal subjects imagining back pain. The invention uses a non-linear technique to demonstrate that the time-series examined are scale free, and derives a single parameter with which each time-series is characterized, permitting a contrast between pain conditions.

### DESCRIPTION OF THE DRAWING

The invention is described with respect to a drawing in several figures, of which:

FIG. 1 is a set of graphs showing scaling properties of pain ratings; shown are pain time-series, showing pain ratings from a chronic low back pain (CLBP) patient (top), from a post-herpetic neuropathy (PHN) patient (middle), and from a healthy subject imagining back pain (bottom)

FIG. 2 is a graph showing a distribution of a scaling exponent of for CLBP and PHN patients, and for normal healthy subjects imagining back pain;

FIG. 3 shows scaling exponents for rated pain and for imaged measurements;

FIG. 4 shows five sample pain ratings from back pain (A) and PHN (B) patients, from healthy subjects imagining back pain (C), and healthy subjects in response to thermal stimulation of the lower back (D);

FIG. 5 shows fractal properties of pain ratings;

FIG. 6 shows power spectra for the same ratings as shown in FIG. 4;

FIG. 7 shows rescale range analysis plots for the same time series shown in FIG. 1; and

FIG. 8 shows distributions of fractal dimension D for various time series.

### DETAILED DESCRIPTION

In one embodiment according to the invention, subjects indicate their level of pain continuously through a linear potentiometer device that is attached to the thumb and index finger of the dominant hand. Voltage output from the finger device is collected and calibrated by a computer running the software LabView (National Instruments, Austin, Tex.). Subjects are seated in front of a computer monitor, which displays the extent of their finger span by a colored bar (y-axis has an intensity scale of 0-100), providing visual feedback of their rating. Ratings are sampled at 10 Hz.

Eleven CLBP (two males, nine females; mean age, 37 years), 15 PHN (seven males, eight females; mean age, 62 years), and 22 normal healthy subjects (ten males, twelve females; mean age, 34 years) participated in a study. The subjects rated their pain, and the temporal fluctuations of their ratings were examined. In five of the CLBP patients the relationship between brain activity and pain ratings was examined. Patients with CLBP fulfilled IASP criteria and were diagnosed in accordance to recent guidelines. Briefly, all CLBP patients had unrelenting pain for more than one year, primarily localized to the lumbosacral region, with or without pain radiating to the leg. No distinction was made between various etiologies of CLBP. Patients with PHN fulfilled IASP criteria and were diagnosed based on standard guidelines. They all had pain along the course of a nerve after the characteristic acute segmental rash of herpes zoster, for more than 3 months. All patients had ongoing pain and most PHN patients also had touch-evoked pain (allodynia). Patients refrained from using analgesics for 24 hours prior to their pain rating sessions.

Subjects were first trained on the use of the finger-span device. To this end they were presented with a bar on the computer monitor that varied in time and instructed to rate its size with the finger-span device, over a 5-minute trial. Only subjects able to follow the fluctuations of the bar at a consistency level that results in correlation coefficient r>0.75 between rating and bar fluctuations were included in the study, within two attempts. Over 90% of subjects achieved this criterion, within two attempts. Patients were then instructed to rate the fluctuations of their ongoing pain, usually for a period of 5-12 minutes, one or more times for each subject. They were instructed that maximum thumb-finger span should be used to indicate maximum imaginable intensity of pain (level 100) while thumb and finger touching should indicate absence of pain (level 0). Healthy normal volunteers were instructed to imagine back pain and rate its fluctuations in time. Some of the normal volunteers were also instructed to generate ‘random movements’ using the finger-span device.

A separate group of healthy subjects (n=6, 2 sessions per subject) rated acute thermal stimulus pain with the finger-span device. Eight noxious thermal stimuli ranging in duration from 10 to 30 seconds were applied to the lower back (baseline 38° C., peak temperatures 46° C. and 48° C., rise rate 20° C./sec.) via a contact probe (1×1.5 cm Peltier device). Durations and intensities of thermal stimulation as well as inter-stimulus intervals (range 30-60 seconds, mean=55 seconds) were presented in a fixed pseudorandom fashion.

The time series were first preprocessed for noise reduction. The potentiometer used in pain rating introduces noise into the time series that does not reflect fluctuation in finger position or perceived pain, since it is at frequencies much higher than the speed at which subjects can control finger movement. Normal filtering or smoothing would introduce false correlations into the data, so to reduce instrument noise, the data were averaged over a non-overlapping window of width 4, effectively reducing the time series length by a factor of 4, and increasing the sampling period of the time series by 4.

Fractal dimension D of the time series was determined with two independent approaches—rescaled range analysis, and calculation of power spectra. Each approach has its unique advantages and assumptions.

Scaling exponents of the time series were then calculated by rescaled range analysis. Rescaled range analysis measures the extent to which the total distance, R, spanned during a fluctuating trajectory depends on the number of steps or time in the trajectory, τ. A characteristic of scale free trajectories is that R and τ obey a power law, i.e. R∝τ^{α}. To empirically determine the scaling exponent α, for a given time series of length N, we determine the average R for different length scale τ subsets of the original sample. The largest time scale possible is when τ=N. We find the length scale, R, by taking the distance between the maximum and minimum values of the sub-series after detrending. We then scale R by the standard deviation, S, of the step size of this sub-series, S, since series with larger step sizes will naturally have a larger length scale, regardless of their scaling properties. For τ=N there is only one sub-series, for τ=N/2 there are two sub-series, etc. We find the average value of R/S for all sub-series at each time scale down to a minimum scale of 8 samples (reflecting 24 samples in the raw time series) and use them to create the ‘scaling plot’ log(R/S) as a function of log (τ). If the time series exhibits scale free fluctuations, this relationship is linear with slope α. The fractal dimension D, is related to the scaling exponent by the relation D=2−α. In most examples of scale-free behavior, the fluctuation is only scale free in a limited scale range. This can be caused by minimum and maximum limits on the values the series can take on, or by a minimum time scale at which fluctuations can occur. The data described here have both of these constraints. For each time series, therefore, a ‘scaling region’ was empirically chosen, namely a span of τ within which the series exhibits the most scale free behavior. The average R/S value at each available τ was used to compute Δ(R/S), the change in R/S between consecutive points. Δ(R/S) divided by Δτ is an instantaneous estimation of the slope of the scaling plot. In a scale free range, there should be a consistent instantaneous slope. To determine systematically the best candidate for the scaling region for each time series, we consider the set of local slopes generated by all possible τ ranges in the scaling plot. For each possible range, the variance of the local slopes is computed. Within certain constraints, the scaling region is chosen as the range with least variance in local slope, i.e. the most consistently linear region of the scaling plot. Once the scaling region is chosen, linear regression is performed on the points in it to deduce the overall slope and scaling exponent α. To determine the significance of the scaling results of these time series, we constructed surrogate data sets for each one. The surrogates were constructed by shuffling the steps between consecutive points in the averaged data. This created, essentially, a random walk time series, which as expected, yielded scaling exponents close to 0.5.

For a given time varying signal, the power spectrum measures power (energy per unit time) of the signal at each frequency. Power spectra were computed with Welch's averaged periodogram method, in Matlab. Time series were first truncated to length 2048 for PHN, imagined pain and thermal pain, and 1024 for back pain, detrended, and then windowed with a Hanning window. The power as a function of frequency is plotted on a log-log scale. The fractal dimension D is related to the slope of the spectrum β such that D=2+¼β.

In most examples of fractal behavior, the fluctuation is scale free only in a limited scale range. For each time series, therefore, we empirically determine the ‘scaling region’ as the span of τ or frequency within which the series exhibits scale free behavior. Typically, the spectra became flatter outside that range. Based on comprehensive inspection of the spectra and scaling plots, initial scaling ranges were established for each data type. For each individual spectrum or scaling plot these ranges were then incrementally reduced until a region of maximal slope and maximal regression coefficient was identified. This region was then used to compute the scaling exponent.

In addition to subject pain ratings, brain imaging was performed. Functional brain images were acquired on a 1.5T Siemens Vision MRI scanner. A standard clinical quadrature head coil was used to image the entire brain. Functional MRI scans were performed using echo planar gradient-echo acquisition sequence (repetition time of 3.5 sec; echo time of 40 msec; matrix of 64×64; field of view of 240 mm; flip angle of 90°; 4 mm thick slices with no gap). During each fMRI scan, 124 brain volumes were acquired over about 7 minutes. A vacuum beanbag was used to immobilize the head.

Five CLBP patients were used in fMRI to determine the correspondence between brain activity scaling properties and the scaling properties reported by CLBP patients in general. The finger-span device was implemented in the scanner just as it was used in the psychophysical testing outside the scanner. The computer screen where the subject viewed his finger-span was back projected into the scanner to provide the participant with continuous visual feedback of the ratings. Just prior to starting an fMRI scan, subjects were instructed to concentrate on his/her ongoing pain and rate it with the finger-span device for the duration of the scan. A trigger pulse from the scanner was used to collect the ratings for every slice acquisition, which provides sampling at 10 Hz.

Preprocessing and data analysis were performed using FEAT software (FMRIB Expert Analysis Tool; http://www.fmrib.ox.ac.uk/fsl, Oxford University, UK). The first four volumes of each run were discarded. Preprocessing included: slice acquisition time correction; head motion correction; spatial smoothing (Gaussian kernel 5 mm FWHM); and a high pass filter (cutoff, 100 sec). A linear regression model was used to describe the data. The covariate of interest was the time series of pain ratings convolved with a gamma-variate hemodynamic response function (width, 3 sec; mean lag, 6 sec). A covariate of no interest was used to further correct head motion artifacts, derived from the motion correction procedure during fMRI data preprocessing. The fMRI signal was linearly modeled on a voxel by voxel basis with local autocorrelation correction. Regression coefficients at each voxel were transformed to Z-scores indicating the statistical significance of pain rating-related fMRI blood oxygenation level dependent signal change. The time curve for the voxel with maximum Z-score was used to calculate its scaling exponent.

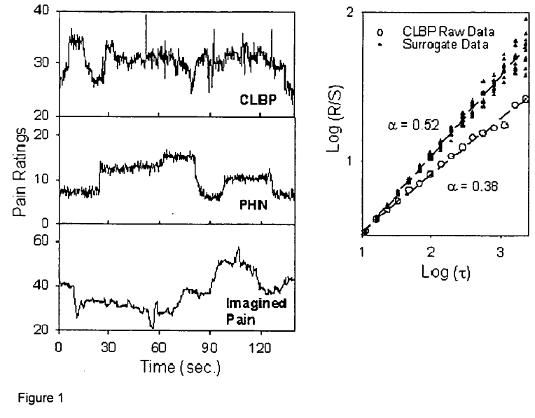

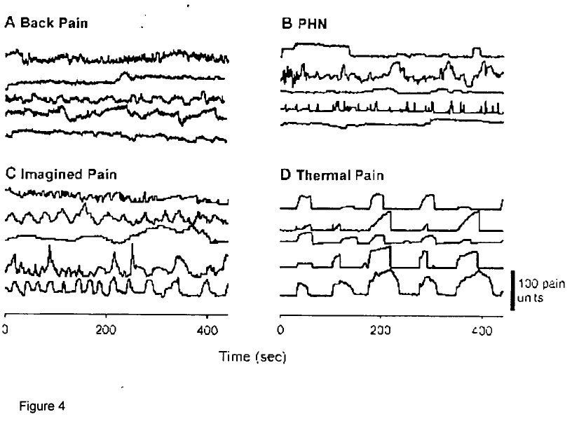

A cursory examination of the time series generated by the patients indicated a lack of periodic oscillations with no obvious sinusoidal frequencies (FIG. 1A). Instead the pain rating fluctuations resemble random time series. Therefore, rescaled range analysis was applied to determine the extent to which these ratings corresponded to random walk type processes. Thermal stimulus ratings were “pulse-like” remaining relatively constant in between the presentation of the painful stimulus (FIG. 4D). Imagined pain ratings (FIG. 4C seem intermediate between thermal pain and patients' ratings in the amount of fluctuations. It may also be observed that the time series for the patients look rougher, that is, they show more non-cyclic fluctuations, or look noisier, than the curves for the normal subjects. These qualitative impressions can be formalized by computing and comparing their fractal dimension D.

Rescaled range analysis indicated that ratings for the first three groups of subjects studied, CLBP, PHN, and healthy subjects imagining pain, were scale free, i.e. had fluctuations at all time scales. This was demonstrated by the presence of a power law relationship between the amplitude of the fluctuation and the length of time τ at which it was measured (FIG. 1b). For pain, “scale free” implies that, for a given time scale τ, the perceived pain will exhibit an average amplitude fluctuation proportional to τ^{α}. In patients with CLBP average scaling exponent α=0.37±0.06, n=11 patients, with mean coefficient of the fit <R2>=0.995 for n=61 ratings; while for PHN α=0.51±0.13, n=15 patients (<R2>=0.997, n=55 ratings). Ratings in healthy subjects who imagined ongoing back pain and reported its fluctuations were also scale free with scaling exponent α=0.69±0.15, n=22 subjects (<R2>=0.998, n=22). For each time series a randomized version of the raw data (surrogate time series) was also generated. Overall, the surrogates give α=0.530±0.033, n=138, corresponding to random walk time series. The demonstration that the pain ratings were scale free confirmed the impression that these ratings do not contain periodic oscillations.

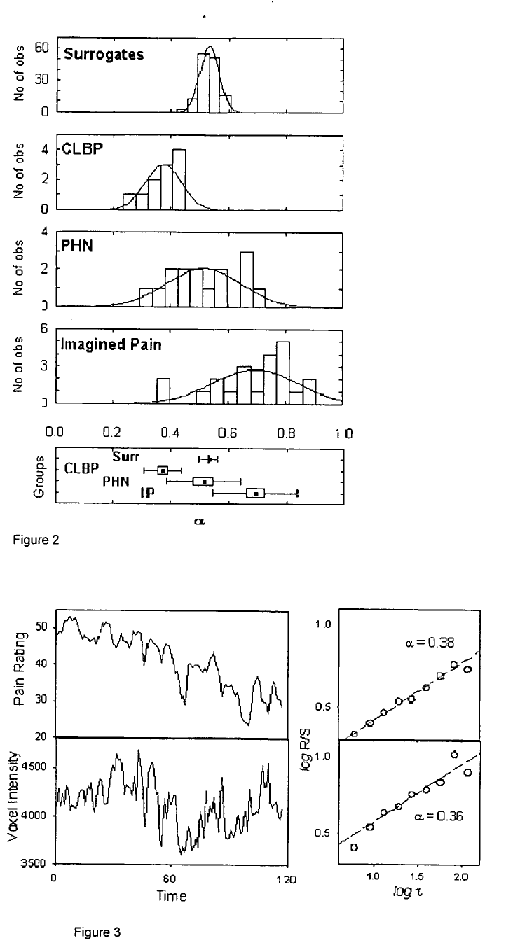

FIG. 2 shows the distribution of α-values for the first three groups studied, in contrast to the distribution for the corresponding surrogates. One-way analysis of variance performed on individual subject α-values showed a highly significant difference across the three groups (CLBP, PHN, imagined pain), F_{2,47}=25.7, P<10⁻⁷. All pairwise planned-comparisons also showed significant differences (F>10.0, P<0.001; for all pairwise comparisons).

An alternative comparison is to contrast the number of ratings that are distinct from their corresponding surrogates. A given scaling exponent α was considered different from its surrogate if α was outside two standard deviations of its 20 surrogates. In CLBP 75% ( 46/61), in PHN 76% ( 42/55), and for imagining pain 86% ( 19/22) of the ratings were distinct from their surrogates.

A subgroup of the normal subjects was also instructed to move their fingers randomly (n=8 subjects). In contrast to the scale free ratings generated for imagining pain, this instruction led to time-series dominated with periodic fluctuations. Rescaled range analysis for these time series indicated that they were not scale free over a sufficient scaling region for computation of α to be meaningful (data not shown).

To test the correspondence between neuronal activity and pain ratings scaling properties were examined for time-course of brain activity in 5 CLBP patients in relation to their pain ratings. In each patient, the brain voxel with maximum Z-score was selected and its scaling exponent computed. Scaling exponent for brain activity was α=0.31±0.05, while for fluctuations in pain rating α=0.39±0.04. An example is shown in FIG. 3, where the gross features of variability of brain activity correspond to the pain rating variability, resulting in similar exponents for both. The peak Z-value (3.74 in FIG. 3) can be viewed as a measure of the strength of the correlation between the pain rating (convolved with hemodynamic response function) and brain activity, which indirectly reflects underlying neuronal activity.

Fractal dimension D of fMRI activity in medial prefrontal cortex (18, 60, 12) was highly correlated to pain rating fractal dimension (r=0.96, P=0.008). Similarly, D for fMRI activity of cingulate at the level of the genu (10, 22, 28) was correlated to D of pain rating (r=0.92, P=0.02). In contrast, D of fMRI activity in temporal cortical regions (−54, −44, −8) (56, −40, 10) were not related to D of pain ratings (r=0.37, P=0.54; and r=0.16, P=0.79). Medial prefrontal cortex and cingulate at the level of the genu are brain regions activated with fluctuations of ongoing pain in back pain patients, while activity in the temporal cortical areas are not related to fluctuations in back pain. Therefore, this analysis demonstrates correspondence between D for brain activity in regions involved in ongoing pain of back pain and D for rating of pain.

As mentioned above, so far as applicants are aware, this study was the first to examine temporal properties of ongoing pain. It was found that subjective reports of fluctuations of ongoing pain possess dynamical properties that can be characterized with a single parameter. This parameter, scaling exponent α, indicates that pain ratings are scale free, that they are distinct for two chronic pain conditions studied here, and cannot be generated by normal subjects imagining pain. In patients where α was examined for pain ratings and the related α for brain activity, a close correspondence was observed between them, implying that the scale free properties of pain variability are a manifestation of brain neuronal activity.

It has also been shown that D is distinct for the two chronic pain conditions studied here, and cannot be mimicked by normal subjects imagining pain. In back pain patients where we examined D for pain ratings and for brain activity, a correspondence was observed between them only for brain regions involved in perception of ongoing back pain, implying that the scale free properties of pain variability are a manifestation of neuronal activity involved in pain perception. These results, therefore, demonstrate that time variability of ongoing pain may be used as an objective metric for documenting the presence, and studying the properties of chronic pain.

Time varying signals have a Euclidean dimension (E) of 1. When they fluctuate non-periodically they can have fractal dimensions spanning between Euclidean dimensions 1-2. The more rough their fluctuations the higher their fractal dimension. As illustrated for pain ratings, fractal time series show power law scaling for variability in time and for power in frequency. Moreover, such time series exhibit self-similarity and ill-defined mean and standard deviations. Many methods have been proposed to estimate the fractal dimension of time series. Here we test for correspondence between time and frequency domain analyses.

Many fluctuations in nature are found to be scale free, and frequently α is different from the trivial ½ generated by a purely independent random walk. Time series are said to be ‘persistent’ when the trajectory during a time period has a higher probability of going in the same direction as in the previous period, resulting in α>½. On the other hand, they are called ‘anti-persistent’ when a subsequent period is more likely to be in the opposite direction than in the preceding one, resulting in α<½. Alternatively, anti-persistent processes are ones with fractal dimensions in the range 2.0>D>1.5; while persistent processes have fractal dimensions in the range 1.0<D<1.5. Thus, anti-persistent processes can be thought of as more space filling than persistent processes. It is clear that as time passes, persistent processes on the average make larger excursions than anti-persistent ones because of the increased tendency to go in the same direction. There is a large spread in the scaling exponents computed for different pain patients. However, CLBP patients show anti-persistence, meaning that on the average more intense pain is followed by weaker pain. PHN patients instead show both anti-persistent and persistent time series. Healthy subjects attempting to imagine fluctuations of pain generate the most persistent ratings, suggesting that normal subjects assume that pain does not undergo much fluctuation. The properties of fluctuations in ongoing pain most likely reflect the interaction between peripheral and central processes inducing the pain with the coping mechanisms that patients develop to deal best with the condition (including use of analgesics and other medications). From this viewpoint the extent to which a given pain rating is anti-persistent may be interpreted as reflecting the ability of the patient to cope with the ongoing pain. One obvious candidate system that can control this parameter is the integrity of descending modulatory pathways, which provide supraspinal feedback control on spinal cord nociceptive neurons and limit nociceptive information transmission cephalad. Relative potentiation of descending inhibitory and facilitatory pathways in different clinical pain conditions would naturally lead to pain ratings with distinct persistent or anti-persistent scaling properties.

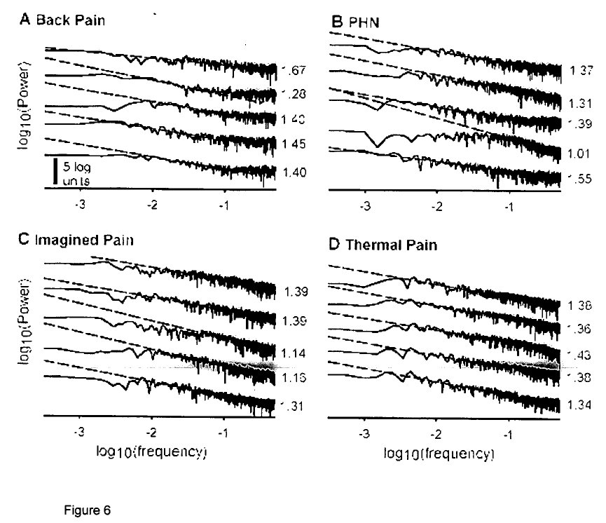

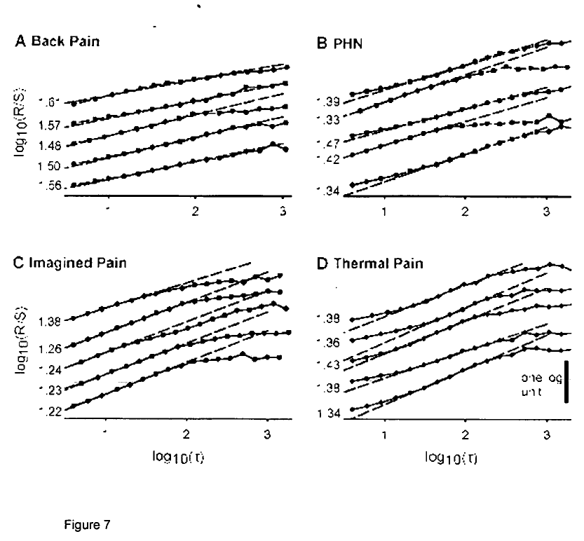

We calculated fractal dimension of the ratings from power spectra and rescaled range analysis. FIGS. 6 and 7 show power spectrum analysis and rescaled range analysis for the corresponding ratings shown in FIG. 4. Resultant D-values are similar between the two methods of measurement, and tend to be higher in the patients. These figures illustrate the range of scales at which power law scaling is exhibited by the two methods. These scale ranges generally correspond between the two methods, and seem to be larger for patients' rating and smallest for thermal pain ratings (more obvious in time domain, FIG. 7D).

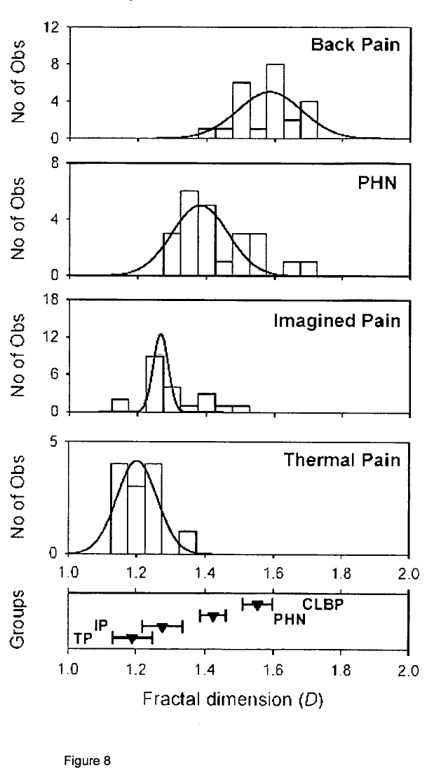

FIG. 8 shows the distribution of D-values for the four groups studied, based on the rescaled range analysis. In back pain patients average fractal dimension D=1.55±0.08 (mean±S.D., n=23); while for PHN patients D=1.42±0.11 (n=58). For ratings in healthy subjects who imagined ongoing back pain and reported its fluctuations D=1.28±0.09 (n=21), and for healthy subjects ratings of thermal pain D=1.19±0.07 (n=12; with mean coefficient of the fit r²>0.99 for all four groups). One-way analysis of variance showed a highly significant difference of D across the four groups (back pain, PHN, imagined pain, thermal pain), F_{3,110}=52.6, P<10₋₇. All pairwise planned-comparisons also showed significant differences, with the smallest difference between imagined pain and thermal pain (F=8.2, P<0.007; all other pairwise comparisons had P<10⁻⁶).

To test the robustness of our results we compare dimensions determined by the two methods. We examine the difference, D determined in time domain by rescale range analysis (Dₜ) minus D determined in frequency domain by power spectrum (D_{f}), Dₜ−D_{f}. The distance from zero of the distribution of this value is used as a measure of confidence regarding fractal properties of the ratings. When a cutoff of two standard deviations is used, only 5 of the ratings fall outside this threshold, and the group means and standard deviations remain unchanged. When a cutoff of one standard deviation is used, then 10 of the 12 thermal pain ratings are excluded, but more than 80% of the other ratings remain below the cutoff. Moreover, the D-values for the three groups, back pain (D=1.53±0.07, n=16), PHN (D=1.42±0.10, n=44), and imaged pain (D=1.26±0.10, n=15), remain essentially unchanged and statistically significantly different from each other.

It is worthwhile to note that the applicants have been using subjective pain ratings in various chronic pain conditions as a tool to study brain activity. To date, over 100 such patients have been asked to rate their ongoing pain and the vast majority readily comply. Only rarely, less than 5%, patients insist that their pain is invariant over the time scale of a few minutes. It is also important to note that the ratings in normal subjects are strongly dependent on the instruction set, and subtle changes in these instructions may produce different scaling exponents. The applicants reasoned that, since back pain is the most common form of sustained pain healthy subjects may experience, imagining back pain by healthy subjects had the best chance of mimicking pain ratings in patients. The volunteers tested readily complied with the instruction and yet resulted in ratings that do not match that of CLBP patients.

The applicants' use of pain ratings in fMRI studies provides a neuronal activity based validation of the approach. Here this idea was directely tested by examining scaling properties of brain activity for regions best related to the pain ratings. A tight relationship was found for scaling exponents between pain ratings and brain activity. Note that an exact relationship is not expected, given the extensive transformations that fMRI data and the vector for which activity is analyzed undergo and, given that fMRI data are contaminated with background noise arising from multiple independent sources.

In summary, nonlinear analysis of pain dynamics over a scale of minutes reveals that perceived pain fluctuates as a scale free process. Patients with CLBP, of various etiologies, show scaling exponents in the anti-persistent range, which may reflect their ability in limiting the amount of pain experienced by activating coping mechanisms. The PHN pain dynamics shows a broader distribution, probably indicating different subtypes (Petersen et al., 2000). The applicants surmise that PHN patients with persistent exponents have a more limited ability to control their pain. The interpretation is consistent with the applicants' preliminary observations: PHN patients who respond to therapy tend to shift their pain-rating time series to a less persistent value. The ratings in subjects asked to imagine pain indicate that their expectations do not match reality. Therefore, the applicants demonstrate that temporal properties of pain can reveal novel information with potential clinical significance as a biomarker.

The figures will now be discussed in greater detail.

FIG. 1: Scaling properties of pain ratings.

(A) Pain time-series, example ratings from a CLBP (top) and PHN (middle) patient, and a healthy subject imagining back pain (bottom). A device attached to the subject's thumb and index finger tracks ratings by producing a signal proportional to finger-span. Maximum finger-span corresponds to maximal pain=100, and zero finger-span, fingers touching, corresponds to no pain=0.

(B) Scaling exponent for the CLBP time-series shown in (A). The mean of log(R/S) as a function of log(τ) for the CLBP time-series is shown (open circles) and the same relationship for ten surrogate time-series (red dots). The linear fits and slopes for the pain rating and its surrogates are also shown, with the resultant slopes that define the scaling exponents (α).

FIG. 2: Distribution of scaling exponent, α, for CLBP and PHN patients, and for normal healthy subjects imagining back pain. Individual subject α-values are plotted, and the fitted normal distribution for each group displayed. The top panel is for surrogates. In this case a single surrogate was generated for each subject's rating, and its distribution and fitted normal distribution plotted. As expected this distribution is centered around α=0.5. The bottom panel compares the four data sets by displaying their mean, +/−1 standard error (boxes), and +/−1 standard deviation (whiskers). Analysis of variance between CLBP, PHN and normal subjects imagining pain (IP) was highly significant (P<10-7).

FIG. 3: Similarity of scaling exponents for pain rating and brain activity. Rating of ongoing pain is used to identify brain activity with fMRI in CLBP patients. Results from one patient are shown. Top left is the patient's rating of ongoing pain during an fMRI scan. Bottom left shows the fMRI signal for the brain voxel with maximum Z-score (3.74), i.e. the voxel best predicting the patient's pain rating. Right panels are corresponding scaling exponents. Time axis is in seconds/3.5.

FIG. 4 shows example pain ratings. Five example ratings from back pain (4A) and PHN (4B) patients, healthy subject imagining back pain (4C), and healthy subjects in response to thermal stimulation to the lower back (4D). Pain ratings (vertically shifted for clarity) are plotted at the same scale (shown by the calibration bar in the bottom right corner).

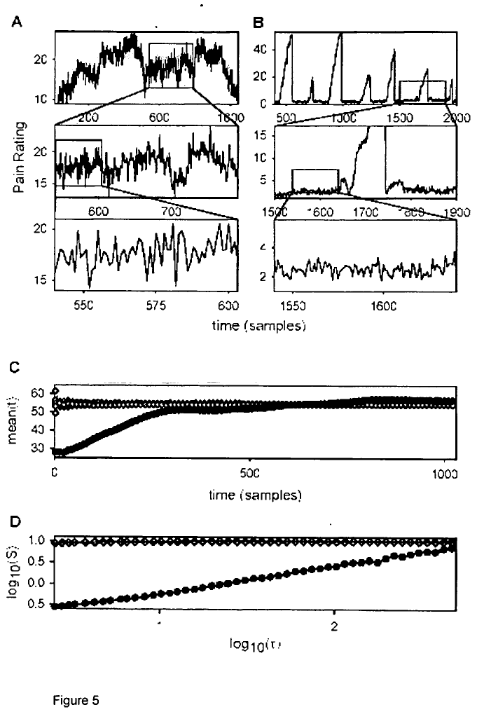

FIG. 5 shows properties of fractal pain ratings.

FIG. 5A shows statistical self-similarity of a fractal pain rating. A back pain patient's pain rating as a function of time in samples (1 sample=400 msec) is shown at three different scales. The general time variability pattern is self-similar at all three magnifications. As the horizontal scale is reduced by a factor of 4, the vertical scale is reduced by a factor of 4_{α}, where α=0.34, indicating power law scaling with fractal dimension D=2-0.34. The boxes on each plot indicate the ranges in the plot below. (Upper plot has scale 1000×18; middle plot has scale 1000*(¼)×18*(¼)_{α}, and bottom plot has scale 1000*( 1/16)×18*( 1/16)_{α}).

FIG. 5B shows pain rating from thermal stimulation. Magnifying a rating that is minimally fractal fails to show self-similarity. Scaled as in A, with α=0.85. Note that vertical range is extremely dependent on exact horizontal location.

FIG. 5C shows that the mean does not converge for fractal pain ratings. Mean of successively longer samples of the pain rating time series are used in A (solid curve). Open symbols show the means of 10 randomly shuffled surrogates, which quickly converge to a mean.

FIG. 5D shows an excess of variance for fractal pain ratings. Average standard deviation, S, for all non-overlapping consecutive sub-samples of size τ as a function of τ is shown for time series in A (solid symbols). Open symbols show the results of same computation for 10 surrogates created by randomly shuffling the points of the original data series (surrogate data are so similar that they fall on top of each other). The fractal pain rating variance does not converge, but continues to increase by a power law as sample length increases (exponent is 0.64). The surrogates, on the other hand, quickly converge to a fixed variance or standard deviation.

FIG. 6 shows power spectra for the same ratings shown in FIG. 4, presented in the same vertical order. Frequency units are in cycles per sample, plotted in log scale. Regression fit lines over the chosen scaling region are also shown (dashed lines). Numbers by each spectrum indicate the fractal dimension D derived from the slope β of the linear fit of the spectrum.

FIG. 7 shows rescale range analysis plots for the same time series shown in FIG. 4, presented in the same vertical order. The mean ratio of the range (R) to the standard deviation (S) at different sample sizes (τ) is shown in log-log plots. Regression lines (dashed lines) were computed from the points in the scaling region. Numbers on each curve indicate fractal dimension D derived from the slope of the (R/S) points in the scaling region. τ is in units of samples.

FIG. 8 shows a distribution of fractal dimension, D, for chronic low back pain (CLBP) and PHN patients, and for normal healthy subjects imagining pain (IM) or rating thermal pain (TH). Spread of D-values and fitted normal distribution for each group is displayed. The bottom panel compares the four data sets by displaying their mean (triangles) and 99% confidence intervals (whiskers). Analysis of variance between the four groups was highly significant (P<10⁻⁷).

Several embodiments of the invention can be described. In one embodiment, a patient is asked to indicate a level of perceived pain by making a physical indication indicative of the level of perceived pain. Data indicative of the physical indication are collected for times during a collection interval. Non-linear scale-invariant behavior is quantified in the collected data, for example by use of a processor executing a stored program. A measure of a presence of pain is arrived at as a function of the quantified non-linear scale-invariant behavior. This measured presence of pain is communicated to a user.

The approach presently thought to be preferable is one on which the physical indication comprises moving fingers of the patient together and apart to indicate the level of perceived pain. It will be appreciated, however, that any of a variety of physical indications by the patient may well serve this purpose, and may well turn out to provide the scale-invariant behavior described.

In the embodiment in which the physical indication comprises finger movements, then before the data are collected, leads are attached to the fingers. In a typical embodiment, the number of fingers is two, and the patient is asked to move the fingers closer together to indicate less perceived pain, and the patient is asked to move the fingers further apart to indicate more perceived pain.

One way to quantify the scale-invariant behavior is to calculate a Hurst exponent for the collected data, in which case the method associates specific ranges of Hurst exponents with specific types of chronic pain, and associates another range of Hurst exponents with a fake pain or non-pain state. For example the method may associate a smaller Hurst exponent with presence of pain.

Another way to quantify the scale-invariant behavior is to calculate a Lyapunov exponent for the collected data, in which case the method associates specific ranges of Lyapunov exponents with specific types of chronic pain, and associates another range of Lyapunov exponents with a fake pain or non-pain state. For example the method may associate a smaller Lyapunov exponent with presence of pain.

The quantification may for example be performed in a processor in a personal digital assistant, or may be performed in a processor in a personal computer.

It is desirable, for each patient, to store arrived-at measures of pain for repeated measurements. This may permit further statistical analysis and identification of trends.

Those skilled in the art will have no difficulty devising myriad obvious variations and improvements upon the invention without undue experimentation, all of which are intended to be encompassed within the scope of the claims which follow.

What is claimed is:

1. A method comprising the steps of:
asking a patient to indicate a level of perceived pain by making a physical indication indicative of the level of perceived pain;
collecting data indicative of the physical indication for times during a collection interval;
by means of a processor, quantifying non-linear scale-invariant behavior in the collected data;
arriving at a measure of a presence of pain as a function of the quantified non-linear scale-invariant behavior; and
communicating the measured presence of pain to a user.

2. The method of claim 1 wherein the physical indication comprises moving fingers of the patient together and apart to indicate the level of perceived pain.

3. The method of claim 2 further comprising the step, performed before the collecting step, of attaching leads to the fingers.

4. The method of claim 3 wherein the number of fingers is two.

5. The method of claim 2 wherein the asking step is further characterized in that the patient is asked to move the fingers closer together to indicate less perceived pain, and the patient is asked to move the fingers further apart to indicate more perceived pain.

6. The method of claim 1 wherein the quantifying step comprises calculating a Hurst exponent for the collected data, and wherein the arriving step comprises associating specific ranges of Hurst exponents with specific types of chronic pain, and associating another range of Hurst exponents with a fake pain or non-pain state.

7. The method of claim 6 wherein the arriving step further comprises associating a smaller Hurst exponent with presence of pain.

8. The method of claim 1 wherein the quantifying step is performed in a processor in a personal digital assistant.

9. The method of claim 1 wherein the quantifying step is performed in a processor in a personal computer.

10. The method of claim 1 further comprising storing, for a patient, arrived-at measures of pain for repeated performances of the asking, collecting, quantifying, and arriving steps.

11. The method of claim 1 wherein the quantifying step comprises calculating a power spectrum for the collected data, and wherein the arriving step comprises associating specific ranges of power spectrum with specific types of chronic pain, and associating another range of power spectrum with a fake pain or non-pain state.

12. Apparatus comprising:
first means measuring a physical indication by a patient;
second means collecting data indicative of the physical indication for times during a collection interval;
third means quantifying non-linear scale-invariant behavior in the collected data;
fourth means arriving at a measure of a presence of pain as a function of the quantified non-linear scale-invariant behavior; and
fifth means communicating the arrived-at measure of the presence of pain external to the apparatus.

13. The apparatus of claim 12 wherein the physical indication comprises moving fingers together and apart to indicate the level of perceived pain.

14. The apparatus of claim 12 further comprising leads for attachment to fingers of the patient.

15. The apparatus of claim 12 wherein the number of leads is two.

16. The apparatus of claim 12 wherein the third means calculates a Hurst exponent for the collected data, and wherein the fourth means associates specific ranges of Hurst exponents with specific types of chronic pain, and associates another range of Hurst exponents with a fake pain or non-pain state.

17. The apparatus of claim 16 wherein the fourth means associates a smaller Hurst exponent with presence of pain.

18. The apparatus of claim 12 wherein the third means comprises a processor in a personal digital assistant executing software.

19. The apparatus of claim 12 wherein the fourth means comprises a processor in a personal computer executing software.

20. The apparatus of claim 12 further comprising sixth means storing arrived-at measures of pain for later retrieval.

21. The apparatus of claim 18 wherein the personal digital assistant communicates wirelessly with a personal computer.

22. The apparatus of claim 12 wherein the third means calculates a power spectrum for the collected data, and wherein the fourth means associates specific ranges of power spectrum with specific types of chronic pain, and associates another range of power spectra with a fake pain or non-pain state.

23. A method comprising the steps of:
asking a patient to indicate a level of perceived pain by making a physical indication indicative of the level of perceived pain;
collecting data indicative of the physical indication for times during a collection interval;
by means of a processor, quantifying a fractal dimension in the collected data;
arriving at a measure of a presence of pain as a function of the quantified fractal dimension; and
communicating the measured presence of pain to a user.

24. The method of claim 23 wherein the physical indication comprises moving fingers of the patient together and apart to indicate the level of perceived pain.

25. The method of claim 24 further comprising the step, performed before the collecting step, of attaching leads to the fingers.

26. The method of claim 24 wherein the number of fingers is two.

27. The method of claim 23 wherein the asking step is further characterized in that the patient is asked to move the fingers closer together to indicate less perceived pain, and the patient is asked to move the fingers further apart to indicate more perceived pain.

28. The method of claim 23 wherein the quantifying step comprises calculating a Hurst exponent for the collected data, and wherein the arriving step comprises associating specific ranges of Hurst exponents with specific types of chronic pain, and associating another range of Hurst exponents with a fake pain or non-pain state.

29. The method of claim 28 wherein the arriving step further comprises associating a smaller Hurst exponent with presence of pain.

30. The method of claim 23 wherein the quantifying step is performed in a processor in a personal digital assistant.

31. The method of claim 23 wherein the quantifying step is performed in a processor in a personal computer.

32. The method of claim 23 further comprising storing, for a patient, arrived-at measures of pain for repeated performances of the asking, collecting, quantifying, and arriving steps.

33. The method of claim 23 wherein the quantifying step comprises calculating a power spectrum for the collected data, and wherein the arriving step comprises associating specific ranges of power spectrum with specific types of chronic pain, and associating another range of power spectra with a fake pain or non-pain state.

34. Apparatus comprising:
first means measuring a physical indication by a patient;
second means collecting data indicative of the physical indication for times during a collection interval;
third means quantifying a fractal dimension in the collected data;
fourth means arriving at a measure of a presence of pain as a function of the quantified fractal dimension;
fifth means communicating the arrived-at measure of the presence of pain external to the apparatus.

35. The apparatus of claim 34 wherein the physical indication comprises moving fingers together and apart to indicate the level of perceived pain.

36. The apparatus of claim 34 further comprising leads for attachment to fingers of the patient.

37. The apparatus of claim 34 wherein the number of leads is two.

38. The apparatus of claim 34 wherein the third means calculates a Hurst exponent for the collected data, and wherein the fourth means associates specific ranges of Hurst exponents with specific types of chronic pain, and associates another range of Hurst exponents with a fake pain or non-pain state.

39. The apparatus of claim 34 wherein the fourth means associates a smaller Hurst exponent with presence of pain.

40. The apparatus of claim 34 wherein the third means comprises a processor in a personal digital assistant executing software.

41. The apparatus of claim 34 wherein the fourth means comprises a processor in a personal computer executing software.

42. The apparatus of claim 34 further comprising sixth means storing arrived-at measures of pain for later retrieval.

43. The apparatus of claim 40 wherein the personal digital assistant communicates wirelessly with a personal computer.

44. The apparatus of claim 34 wherein the third means calculates a power spectrum for the collected data, and wherein the fourth means associates specific ranges of power spectra with specific types of chronic pain, and associates another range of power spectra with a fake pain or non-pain state.
